(19) United States
(12) Patent Application Publication
(10) Pub. No.: US 2018/0214694 A1
(43) Pub. Date: Aug. 2, 2018

(54) Wearable Implantable Medical Device Controller

(71) Applicant: Boston Scientific Neuromodulation Corporation, Valencia, CA (US)

(72) Inventor: Jordi Parramon, Valencia, CA (US)

(21) Appl. No.: 15/883,630

(22) Filed: Jan. 30, 2018

Related U.S. Application Data
(60) Provisional application No. 62/453,412, filed on Feb. 1, 2017.

(51) Int. Cl. A61N 1/36; A61N 1/372

(52) U.S. Cl. CPC A61N 1/36071; A61N 1/37247; A61B 5/024; A61N 1/37217; A61N 1/36139

(57) ABSTRACT

A miniaturized controller includes communication and control circuitry for monitoring and controlling an implantable medical device (IMD). The miniaturized controller includes a display that allows it to essentially mimic the IMD control functionality of a traditional IMD controller. The size of the miniaturized controller, which may be approximately 1.1 cubic inches, enables it to be carried discreetly by a patient during the patient's daily activities. While the miniaturized controller functions as a standalone IMD controller in a first mode of operation, it is also wearable by the patient to function as a smartwatch, for example, in a second mode of operation. In the second mode of operation, the miniaturized controller, which may include sensors for measuring physiological parameters of the patient as well as patient motion when worn by the patient, is capable of providing closed-loop control of the IMD.

### CROSS-REFERENCE TO RELATED APPLICATIONS

This is a non-provisional of U.S. Provisional Patent Application Ser. No. 62/453,412, filed Feb. 1, 2017, to which priority is claimed, and which is incorporated herein by reference in its entirety.

### FIELD OF THE TECHNOLOGY

The present application relates to a controller for an implantable medical device. In particular, the application relates to a miniaturized controller that is also useable as a wearable device.

### INTRODUCTION

Implantable stimulation devices deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and Deep Brain Stimulators (DBS) to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability with any Implantable Pulse Generator (IPG) or in any IPG system.

As shown in FIG. 1, a traditional SCS system includes an Implantable Pulse Generator (IPG) 10 (an Implantable Medical Device (IMD), more generally), which includes a biocompatible device case 12 formed of titanium, for example. The case 12 typically holds the circuitry and battery 14 (FIG. 2) necessary for the IPG 10 to function, which battery 14 may be either rechargeable or primary (non-rechargeable) in nature. The IPG 10 is coupled to electrodes 16 via one or more electrode leads 18 (two of which are shown). The proximal ends of the leads 18 include electrode terminals 20 that are coupled to the IPG 10 at one or more connector blocks 22 fixed in a header 24, which can comprise an epoxy, for example. Contacts in the connector blocks 22 make electrical contact with the electrode terminals 20 and communicate with the circuitry inside the case 12 via feedthrough pins 26 passing through a hermetic feedthrough 28 to allow such circuitry to provide stimulation to or monitor the various electrodes 16. In the illustrated system, there are sixteen electrodes 16 split between two leads 18, although the number of leads and electrodes is application specific and therefore can vary. In a traditional SCS application, two electrode leads 18 are typically implanted on the right and left side of the dura within the patient's spinal column.

FIG. 2A shows a traditional external controller 40 that may be used to control and monitor the IPG 10 via a bidirectional wireless communication link 42 passing through a patient's tissue 5. The external controller 40 may be used, for example, to adjust a stimulation program that is executed by the IPG 10 to provide stimulation to the patient. The stimulation program may specify a number of stimulation parameters, such as which electrodes are selected for stimulation; whether such active electrodes are to act as anodes or cathodes; and the amplitude (e.g., current), frequency, and duration of stimulation at the active electrodes, assuming such stimulation comprises stimulation pulses as is typical.

The IPG 10 of FIG. 2A has a coil antenna 32 to enable bi-directional communications 42 with a cooperative coil antenna 44 in the external controller 40 via near-field magnetic induction. The transmitting coil antenna (32 or 44) generates a magnetic field 42 modulated with data. Such modulation can occur for example using Frequency Shift Keying (FSK), in which ‘0’ and ‘1’ data bits comprise frequency-shifted values (e.g., f0=121 kHz, fl=129 kHz) with respect to the center frequency of the magnetic field 42 (e.g., fc=125 kHz). The modulated magnetic field 42 induces a current in the receiving coil antenna (44 or 32), and is demodulated in the receiving device to recover the data. The magnetic field 42 can comprise a frequency of 10 MHz or less and can communicate over distances of 12 inches or less for example. The coil antenna 32 is depicted inside the case 12 in FIG. 2, but it may also be mounted in the IPG's header 24.

In addition to the communication coil 32, the IPG 10 contains a charging coil 30 for wireless charging of the IPG's battery 14 using an external charging device (not shown), assuming that the battery 14 is a rechargeable battery. If the IPG 10 has a primary battery 14, the charging coil 30 in the IPG 10 and the external charger can be eliminated. The IPG 10 also contains control circuitry such as a microcontroller 34, and one or more Application Specific Integrated Circuit (ASICs) 36, which can be as described for example in U.S. Pat. No. 8,768,453. ASIC(s) 36 can include stimulation circuitry for providing stimulation pulses at one or more of the electrodes 16 and may also include telemetry modulation and demodulation circuitry for enabling bidirectional wireless communications at the coil 32, battery charging and protection circuitry coupleable to the charging coil 30, DC-blocking capacitors in each of the current paths proceeding to the electrodes 16, etc. Components within the case 12 are integrated via a printed circuit board (PCB) 38. While separate communication and charging coils 32 and 30 are shown, a single coil could be used in the IPG 10 for both charging and data telemetry functions, as disclosed in U.S. Patent Publication 2010/0069992.

FIG. 2B illustrates an alternate arrangement in which the IPG 10′ and the external controller 40′ include short-range RF antennas 32′ and 44′, respectively, to enable bi-directional communications via far-field electromagnetic waves 42′. Such communications can occur using well-known short-range RF standards, such as Bluetooth, BLE, NFC, Zigbee, WiFi, and the Medical Implant Communication Service (MICS). The IPG short-range RF antenna 32′ and modulation/demodulation circuitry to which it is coupled would in this case be compliant with one or more of these standards. Short-range RF antennas 32′ and 44′ can comprise any number of well-known forms for an electromagnetic antenna, such as patches, slots, wires, etc., and can operate as a dipole or a monopole, and with a ground plane as necessary (not shown). The short-range RF link 42′ can comprise a frequency ranging from 10 MHz to 10 GHz or so and can communicate over distances of 50 feet or less, for example.

Traditional controllers such as controller 40 are often bulky, hand-holdable devices. The controller 40, for example, includes a display 50, such as an LCD display, for indicating information to a patient. The controller 40 additionally includes multiple buttons to allow control of the IPG 10, such as buttons 52, 54, 56, and 58, as well as ports (not shown) for connecting the controller 40 to a power source or a programming source. These features tend to increase the size and weight of the controller 40. As a result, while traditional controllers enable a patient to adjust stimulation provided by the patient's IPG 10 in a way that many patients find necessary to provide complete pain control, they can be inconvenient for a patient to carry during the course of a day.

To address this inconvenience, miniaturized controllers that can be carried conveniently and discreetly have been developed. An example of a miniaturized controller 80 is illustrated in FIG. 3. Due to the decreased size of the miniaturized controller 80 as compared to the traditional controller 40, the miniaturized controller 80 includes only a subset of the functionality of the traditional controller 40. While the miniaturized controller 80 offers more limited functionality than the traditional controller 40, it enables patients to make the most common types of stimulation adjustments, which include adjusting the strength of stimulation (i.e., increasing or decreasing the current amplitude), selecting a stimulation program (i.e., toggling between pre-configured stimulation programs that use electrodes 16 in different arrangements), and turning stimulation on and off.

The miniaturized controller 80 is small and light enough to be conveniently carried in a pocket or purse, or carried on a keychain or other similar device, but is large enough to be easily handled by a patient with limited hand flexibility. For example, the miniaturized controller may include a housing 88 that is approximately 8.0 cm (3.15 in.) long, 3.5 cm (1.38 in.) wide, and 1.3 cm (0.51 in.) thick. Button 82 decreases the amplitude of the stimulation, while button 83 increases the amplitude. Button 84 turns the IPG 10 on or off. For protection against inadvertently turning the IPG 10 on or off, the button 84 can be recessed a small amount relative to a surface of the housing 88 and generally rounded with a diameter of about 10 mm. Slide switch 86 provides the patient the ability to toggle between pre-configured stimulation programs for the IPG 10 by sliding the switch from one position to another. In the example illustrated in FIG. 3, the slide switch 86 has two positions, one for a first stimulation program and the other for a second stimulation program, allowing the patient to choose between the two programs easily. The positions of the switch 86 are labeled 1 and 2 to indicate the program selected.

Note that the miniaturized controller 80 does not include an LCD display like the controller 40. Instead, the miniaturized controller 80 includes an indicator light 85, which may be a multi-colored LED, for example. The miniaturized controller 80 may manipulate the color and state (e.g., solid, slow flash, fast flash, etc.) of the indicator light 85 to indicate certain conditions such as the successful receipt of a message by the IPG 10 from the controller 80, a failure to communicate a message from the controller 80 to the IPG 10, or a low level of charge of the IPG 10's battery 14. While the indicator light 85 may provide different statuses of the IPG 10 and/or controller 80, these indications are obviously more limited than those which can be provided via the LCD display of the traditional controller 40, for example. Additional details regarding miniaturized remote controllers such as controller 80 are disclosed in U.S. Patent Application Publication 2010/0318159.

While the miniaturized controller 80 is an improvement over a traditional controller 40 in terms of its portability, it would be beneficial to provide a miniaturized controller that included additional functionality.

### BRIEF DESCRIPTION OF THE DRAWINGS

FIG. 1 shows an implantable pulse generator (IPG) in accordance with the prior art.

FIG. 2A shows a cross section of the IPG of FIG. 1 as implanted in a patient, as well as a traditional external controller for communicating with the IPG via inductive magnetic coupling in accordance with the prior art.

FIG. 2B shows a cross-section of an alternative IPG as implanted in a patient, as well as an alternative version of a traditional external controller for communicating with the alternative IPG via short-range RF communications in accordance with the prior art.

FIG. 3 shows a miniaturized controller for communicating with an IPG implanted in a patient in accordance with the prior art.

FIG. 4A-4D show perspective, top plan, bottom plan, and side views of an improved miniaturized controller in accordance with an embodiment of the invention.

FIG. 5 shows the mating of the miniaturized controller with a complementary watchband and the miniaturized controller's functionality as a smartwatch/wearable sensor in accordance with an embodiment of the invention.

FIGS. 6A-6F show various interfaces of the miniaturized controller for performing various IPG control operations in accordance with an embodiment of the invention.

FIG. 7 is a block diagram that shows certain internal components of the miniaturized controller as well as internal components of the IPG utilized for communicating with the miniaturized controller in accordance with an embodiment of the invention.

FIG. 8 shows the ability of the miniaturized controller to provide closed loop control of an IPG when worn by a patient in the smartwatch/wearable sensor configuration in accordance with an embodiment of the invention.

FIG. 9 shows the mating of the miniaturized controller with a band for wearing the miniaturized controller as a necklace in accordance with an embodiment of the invention.

FIG. 10 shows the mating of the miniaturized controller with a band for wearing the miniaturized controller as an anklet in accordance with an embodiment of the disclosure.

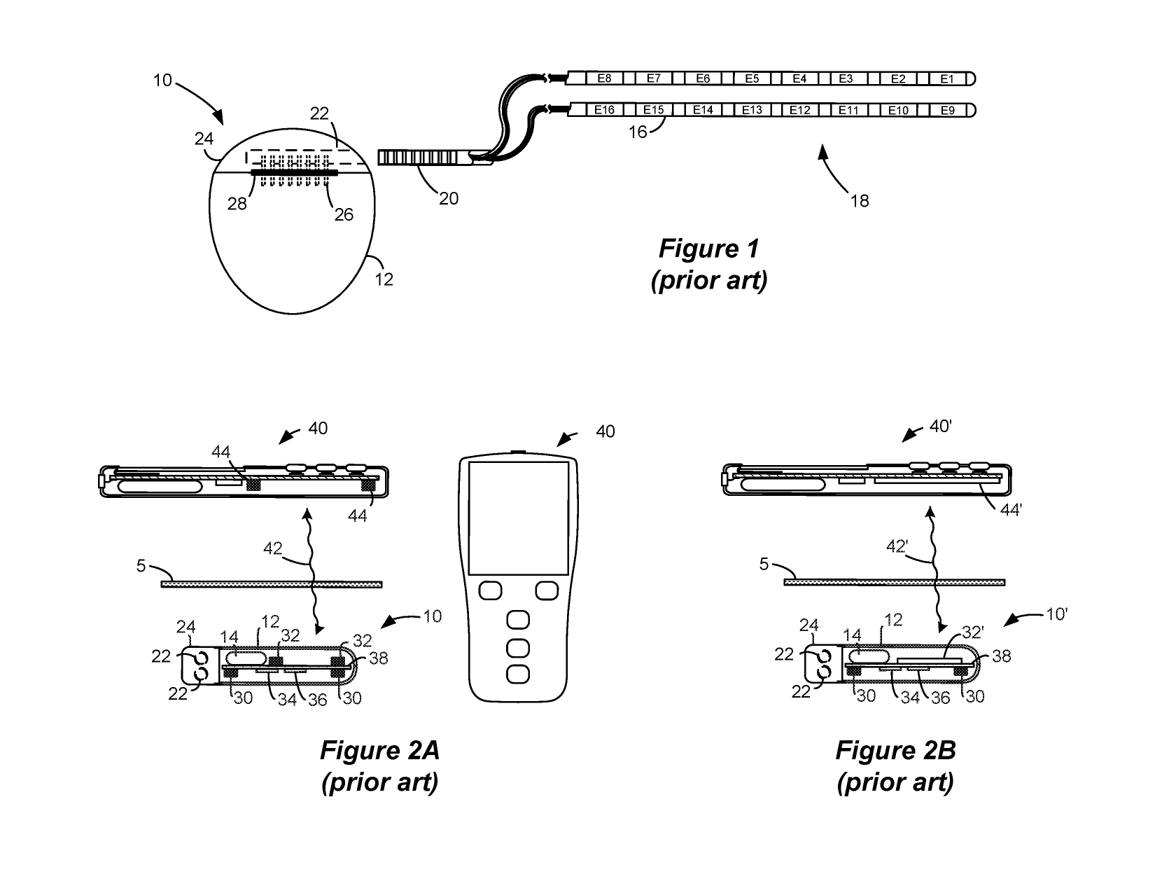

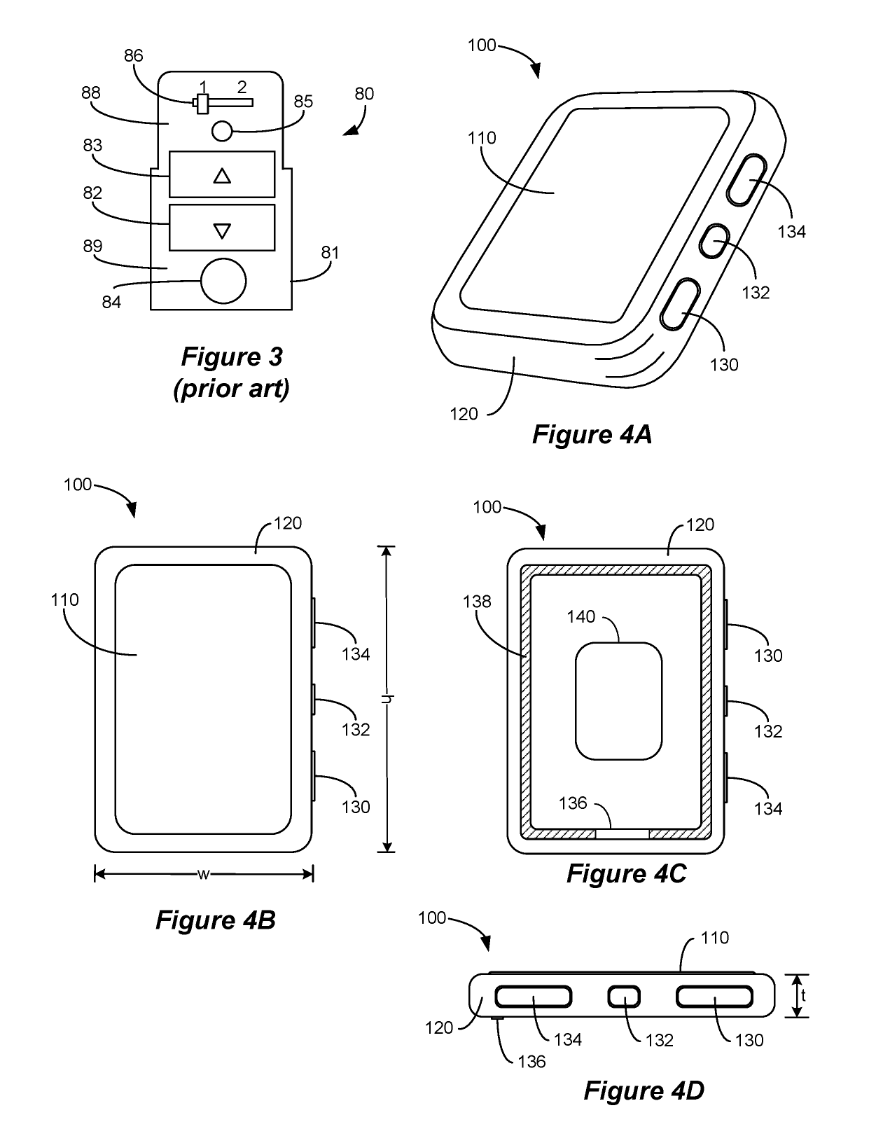

### DETAILED DESCRIPTION

FIGS. 4A through 4D show perspective, top plan, bottom plan, and side views of an improved miniaturized controller 100. The miniaturized controller 100 is utilizable as a standalone controller of an IPG 10 in a first mode of operation (i.e., the controller can act independently to control and/or monitor the IPG 10), and it is additionally attachable to one or more bands (e.g., a watchband) such that the controller 100 is wearable by the patient (i.e., attachable to a body part of the patient) in a second mode of operation. The controller 100 retains its IPG control functionality and may additionally function as a smartwatch and/or wearable sensor in the second mode of operation. As set forth below, the miniaturized controller 100 offers additional functionality as compared to the prior art miniaturized controller 80 as well as closed-loop control functionality that cannot be provided by a full-size traditional controller such as controller 40.

The circuitry that provides the functionality of the controller 100 (described below) is contained within a housing 120. The housing 120 includes openings for the controller 100's display 110; buttons 130, 132, and 134; mode switch 136; and biological/chemical sensor 140. In one embodiment, the housing 120 may have a height h of approximately 1.5 to 2.0 inches, a width w of approximately 1.3 to 1.4 inches, and a thickness t of approximately 0.4 inches. Thus, the housing 120 may have a volume of less than or equal to about 1.1 cubic inches.

The housing 120 may be formed as a single component, and the internal circuitry may be inserted into the housing 120 through the opening in which the display 110 and/or the biological/chemical sensor 140 are ultimately positioned. Alternatively, the housing 120 may be formed from multiple components that are eventually affixed to one another such as via welding or other mechanical fixation to form the housing 120. In one embodiment, the housing 120 is formed from a metallic material such as aluminum, platinum, titanium, gold, or steel. Such metallic materials may be formed into the shape of the housing 120 through casting, forging, and/or precision milling. Alternatively, the housing 120 may be formed of a material such as carbon fiber or a polymer, which may be molded to create the desired shape of the housing 120.

The display 110 may be an LCD or OLED display, and it may be configured as a touchscreen. The display 110 enables the presentation of essentially the same information as can be presented using the traditional controller 40 (although typically through more numerous and simpler interfaces). Thus, the controller 100 is not limited in its ability to present information to a patient in the same way as the miniaturized controller 80. The buttons 130, 132, and 134 protrude through openings in the side of the housing 120, and they can be utilized to control different aspects of the operation of the IPG 10. Because different operating modes can be presented to the patient via the display 110, the buttons 130, 132, and 134 are not limited to a dedicated function. That is, the buttons 130, 132, and 134 may perform different functions based on the information that is presented on the display 110 as described below.

The bottom surface of the miniaturized controller 100 (i.e., the surface opposite the display 110) includes a biological/chemical sensor 140 (although referred to in the singular, the sensor 140 may include multiple sensors that evaluate different physiological parameters of the patient). The sensor 140 is used to evaluate physiological parameters such as a patient's heart rate (or rate variability), blood oxygen saturation, skin temperature, oxygen uptake, respiration, and/or motor neuron activity when the sensor 140 is placed close the patient's skin (e.g., the wrist, chest, or fingertip) in the second mode of operation. The sensor 140 may also evaluate a patient's voice. The sensor 140 may be an optical sensor that includes one or more optical sources for emitting infrared and/or visible light and one or more receivers for detecting infrared and/or visible light. As is known, such optical sensors (e.g., pulse oximeters) emit light into a patient's skin and observe the amount of infrared and/or visible light that is absorbed by the blood flowing below the skin's surface in proximity to the sensor to detect the oxygen saturation and/or heart rate of the patient. The sensor 140 may also measure electrical activity associated with heart contraction in order to detect the patient's heart rate. The sensor 140 may additionally or alternatively include a temperature-measuring device to measure the temperature of the patient's skin. The sensor 140 may additionally or alternatively include electromyography electrodes that measure electrical activity associated with skeletal muscles.

The bottom surface of the controller 100 additionally includes a connector 138, which, in the illustrated embodiment is a groove. The connector 138 is configured to receive a corresponding connector 238 of a watchband 220 that is compatible with the miniaturized controller 100 in order to convert the miniaturized controller 100 from the first mode of operation as a standalone IPG controller to the second mode of operation as a smartwatch/wearable sensor 200.

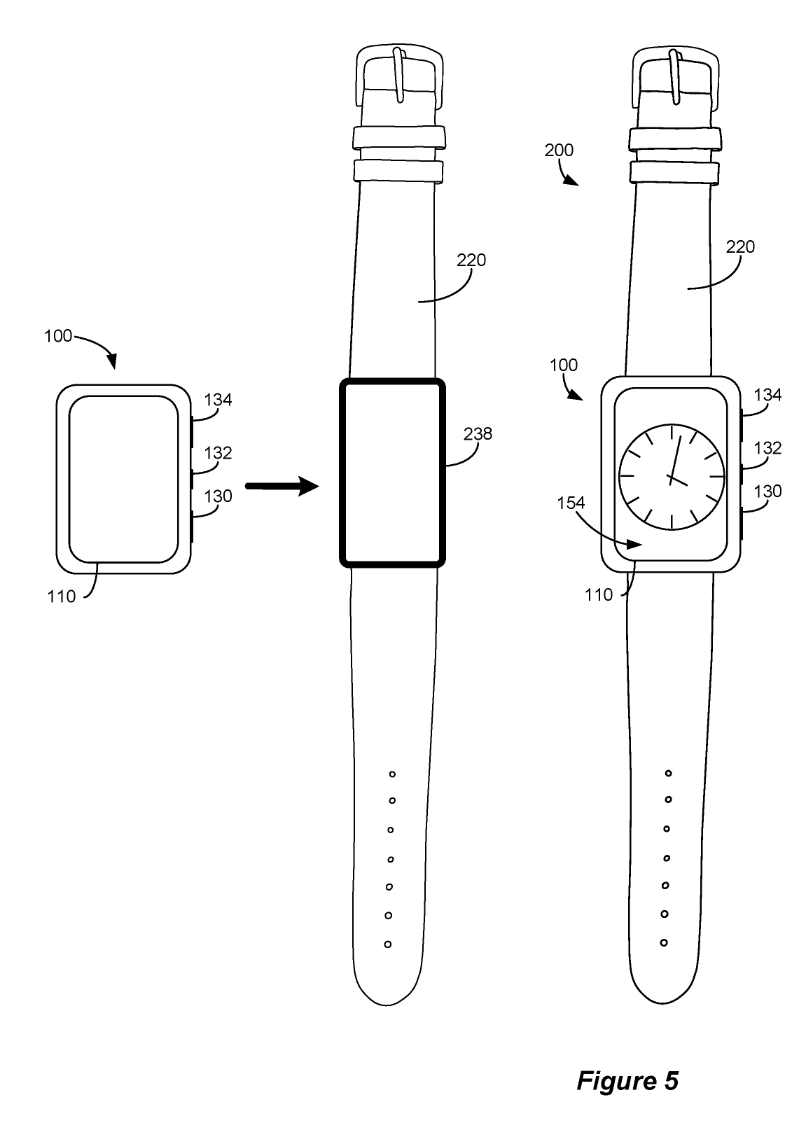

As illustrated in FIG. 5, the miniaturized controller 100 may be affixed to a compatible watchband 220 by aligning the watchband's connector 238 with the miniaturized controller 100's corresponding connector 138 such that the controller 100 can be attached to the patient's wrist. The connector 138 and the connector 238 may be shaped in such a way that the connector 238 is retained within the connector 138 unless a significant force is applied to separate the controller 100 from the watchband 220. For example, the connector 138 may be wider at its bottom than it is at the surface of the housing 120, and the connector 238 may have a corresponding profile such that it is maintained within the connector 138. Alternatively, the controller 100 may be affixed to the watchband 220 via a magnetic attraction between the connector 238 and the connector 138. Note that the connector 238 is preferably open such that the sensor 140 is positioned against the patient's skin (i.e., against the patient's wrist) when the controller 100 is attached to the patient's wrist via the watchband 220.

When the controller 100 is affixed to the watchband 220, the insertion of the connector 238 within the connector 138 toggles a mechanical mode switch 136 positioned within the connector 138 from a first position to a second position. The status of the mode switch 136 is monitored by the internal control circuitry of the controller 100 in order to evaluate whether the watchband 220 is attached to the controller 100, which provides an indication as to whether the controller 100 is being used in the first mode of operation as a standalone controller (i.e., the mode switch 136 is in the first position) or in the second mode of operation as a smartwatch/wearable sensor 200 (i.e. the mode switch 136 is in the second position). As illustrated in FIG. 5, when the mode switch 136 is in the second position indicating that the controller 100 is coupled to the watchband 220, the controller 100's control circuitry may cause the display 110 to present a watch interface 154. In another embodiment, the status of the mode switch 136 may be utilized by the controller 100's control circuitry in combination with signals from the sensor 140 (e.g., signals indicative of skin temperature, heart rate, etc.) to determine whether the controller 100 is being utilized in the second mode of operation.

The controller 100, however, is not limited to displaying a watch interface 154 when it is being used as a smartwatch. In addition to communicating with an IPG 10, the controller 100's communication circuitry enables communication with a base station (e.g., a router, modem, cell tower, laptop, tablet, mobile phone, etc.) that ultimately enables communication with an Internet server. Thus, like existing smartwatches, the controller 100 may be capable of executing software applications to perform such functions as receiving and displaying text messages and emails, receiving and displaying news and weather reports, and accessing and displaying traffic and navigation information, for example. In addition, while the controller 100 may default to the smartwatch interface 154 when it is affixed to the watchband 220, it is preferably still capable of functioning as an IPG controller. For example, the IPG control functionality of the controller 100 may be embodied in a software application that is one of a number of applications that is capable of being executed by the patient when the controller 100 is being used in the second mode of operation. Conversely, when the controller 100 is not affixed to the watchband 220, the controller 100 preferably operates solely as an IPG controller (i.e., additional software applications available in the second mode of operation are not available in the first mode of operation). In one embodiment, the IPG control functionality of the controller 100 is continuously executed as a background operation even when other software applications are being executed by the controller 100 in the second mode of operation. In such an embodiment, the IPG control functionality may take precedent over any other functionality such that any critical alerts related to the operation of the IPG are communicated to the patient regardless of any other use of the controller 100.

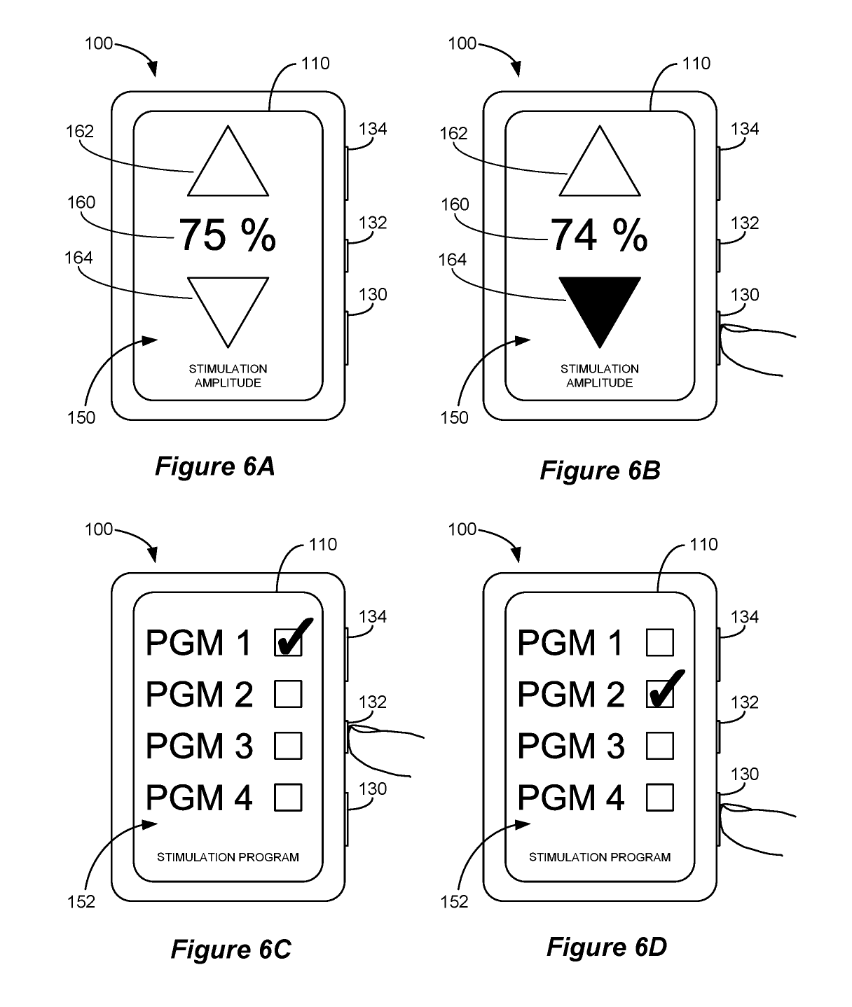

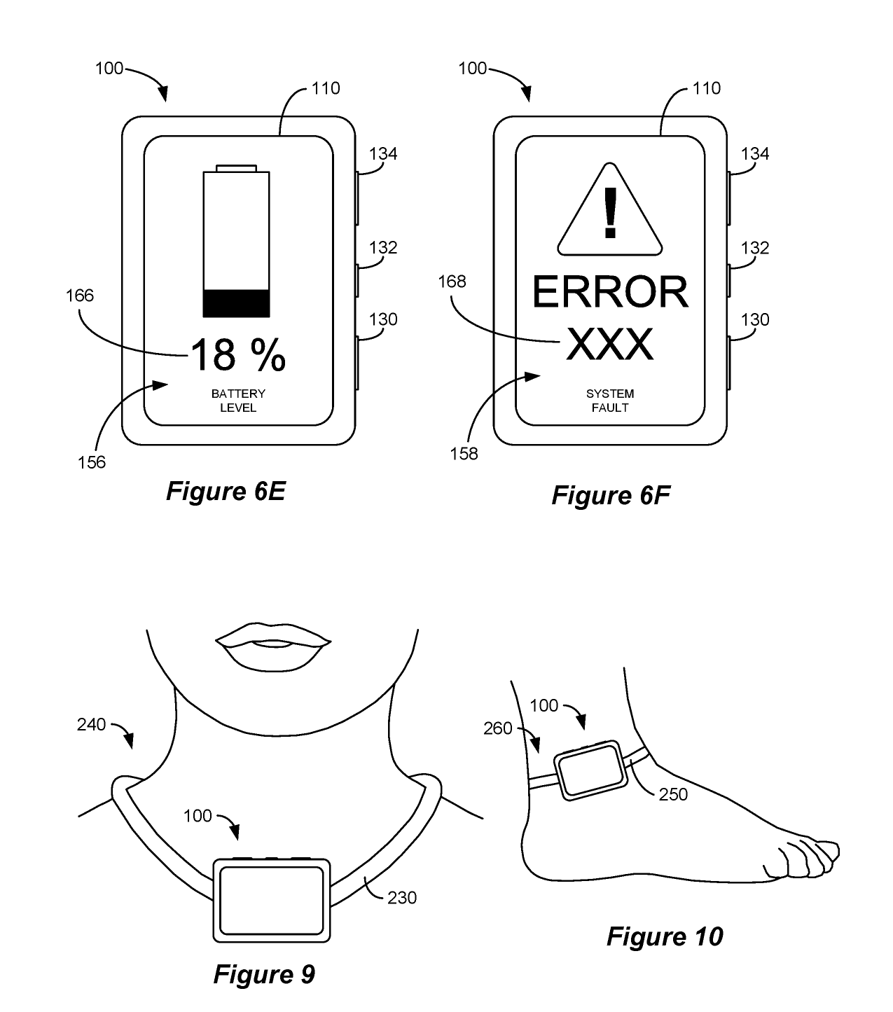

FIGS. 6A through 6F show different interfaces provided on the display 110 when the controller 100 is being utilized for IPG control, which, as noted above, may occur when the controller 100 is being utilized in either the first or second mode of operation. FIG. 6A shows a stimulation amplitude interface 150 that allows the patient to view and adjust the amplitude of the stimulation that is being provided by the IPG 10. An IPG 10 is typically “fitted” to a patient by configuring the IPG with several stimulation programs that alleviate the patient's pain. A stimulation program specifies, among other things, a baseline stimulation amplitude and an allocation of stimulation current across a selected group of the electrodes. By way of example, a stimulation program may specify a baseline stimulation amplitude of 5 mA and an electrode configuration in which current is sourced from electrode E1 and sunk equally from electrodes E10 and E12. While the preconfigured stimulation programs are selected because the combination of stimulation amplitude and the allocation of current across the selected electrodes alleviates a patient's pain, it is typical for a patient to require adjustments to the amplitude in order to achieve a more complete management for different conditions (e.g., activity, posture, time of day, etc.).

The stimulation amplitude interface 150 is a relatively simple interface that enables the patient to make these types of changes. The interface 150 provides an indication 160 of the current stimulation amplitude and arrows 162 and 164 that enable the patient to increase or decrease the stimulation amplitude and provide a visual confirmation of the patient's request to make such an adjustment. The indication 160 of the current stimulation amplitude is expressed as a percentage of the baseline stimulation amplitude of the stimulation program that is being executed by the IPG 10. Based on the above example stimulation program, the 75% indication 160 in FIG. 6A indicates that 3.75 mA is being sourced from electrode E1 (i.e., 75% of the baseline stimulation amplitude of 5 mA) and 1.875 mA is being sunk from each of electrodes E10 and E12.

FIG. 6B illustrates the adjustment of the stimulation amplitude via the interface 150. More specifically, FIG. 6B illustrates the use of the button 130 to decrease the stimulation amplitude. The patient's request to decrease the stimulation amplitude is acknowledged by the filled arrow 164. Button 134 may be similarly used to increase the stimulation amplitude. In the illustrated embodiment, the granularity of the amplitude adjustment is set to 1%, but the adjustment could also be configured to be more or less granular. While the buttons 130 and 134 may be utilized to adjust the stimulation amplitude as shown, if the display 110 is a touchscreen, the arrows 162 and 164 may be touched to increase and decrease the stimulation amplitude.

FIG. 6C illustrates a stimulation program selection interface 152. As described above, the IPG 10 is usually “fitted” to a patient by establishing a set of preconfigured stimulation programs. Each of these programs may provide electrical stimulation in different areas (i.e., using different electrodes), and the patient may switch between these programs to identify the stimulation program that provides the most complete pain relief at a given time. The stimulation program selection interface 152 enables the patient to toggle between the preconfigured stimulation programs. FIG. 6C illustrates the use of the button 132 to switch from the stimulation amplitude interface 150 to the stimulation program selection interface 152. Alternatively, if the display 110 is a touchscreen display, a touch gesture such as a swipe may be utilized in lieu of, or, in addition to, the button 132 for switching between interfaces. When the stimulation program selection interface 152 is displayed, the patient can toggle through a list of the preconfigured stimulation programs using the buttons 130 and 134 (to move “down” and “up” through the list, respectively), or, can select the desired program via a touchscreen interface if available.

FIG. 6D illustrates the use of the button 130 to switch the stimulation program from the first preconfigured program “PGM1” to the second preconfigured program “PGM2.” While four preconfigured stimulation programs are shown, more or fewer stimulation programs may be configured and displayed via the interface 152. In addition, while the stimulation programs are illustrated as having generic numeric designations, the preconfigured stimulation programs may alternatively be given a more descriptive label (e.g., upper leg pain, lower back pain, etc.), which label may be depicted via the stimulation program selection interface 152.

FIG. 6E illustrates a battery level interface 156. The battery level interface 156 displays the charge level 166 (as a percentage of the maximum charge) of the IPG's battery 14. The battery level interface 156 can be accessed in the same way as the other IPG control interfaces (e.g., using the button 132 or a swipe gesture on a touchscreen). When the battery level interface 156 is accessed in this manner, the controller 100 may poll the IPG 10 to retrieve the current charge level of the battery 14. Alternatively, the IPG 10 may routinely communicate the charge level of its battery 14 to the controller 100. In one embodiment, the battery level interface 156 may be displayed in response to the receipt of a communication from the IPG 10 indicating that the charge level of the battery 14 is below a threshold value (e.g., 20%).

FIG. 6F illustrates a system fault interface 158. The system fault interface 158 shows an error code 168 for any active errors in the IPG 10. A short description of the type of error may also be displayed via the interface 158. The error code 168 can be utilized to determine the type of error and the appropriate response, which response may be performed by the patient, a clinician, or a representative of the manufacturer of the IPG 10 based on the type of error. The system fault interface 158 can be accessed in the same way as the other interfaces (e.g., using the button 132 or a swipe gesture on a touchscreen), or it may only be displayed when the controller 100 receives a communication from the IPG 10 indicating the occurrence of an error. FIGS. 6A through 6F illustrate several example IPG control interfaces, but it will be understood that different and/or additional interfaces (e.g., an interface to power the IPG 10 on and off) may also be provided.

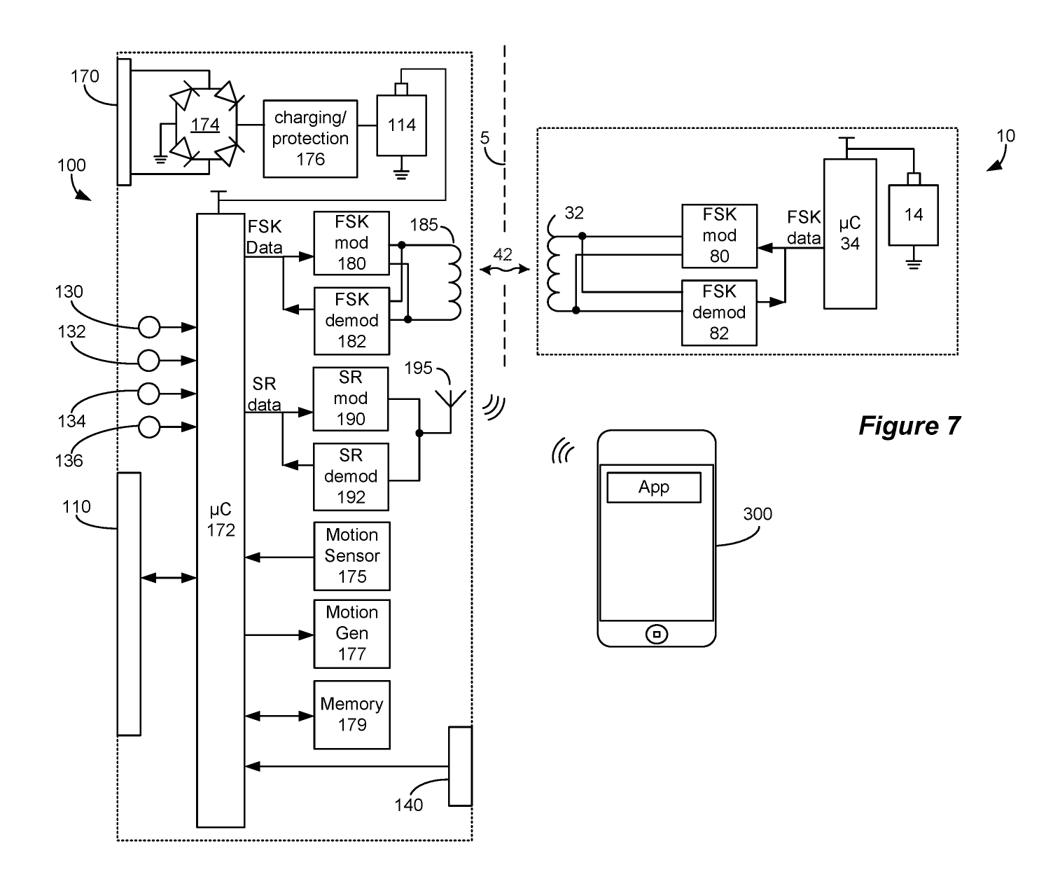

FIG. 7 is a block diagram that shows the connectivity of the internal components of the controller 100 along with the internal components of the IPG 10 utilized for communicating with the controller 100 (additional components of the IPG 10 are omitted). As illustrated, the controller 100's operating power is supplied by a battery 114. In the illustrated embodiment, the battery 114 is a rechargeable battery. The rechargeable battery 114 is charged via energy provided at a port 170, which may be a connector for receiving power directly from a connected charger or a coil for receiving energy through magnetic induction. In either case, AC current received at the port 170 is rectified (174) to DC levels, and used to recharge the battery 114, perhaps via a charging and battery protection circuit 176 as shown. Although a rechargeable battery is illustrated, the battery 114 may also be non-rechargeable, in which case the port 170, rectifier circuitry 174, and charging and battery protection circuit 176 may be omitted.

The battery 114 powers the controller 100's control circuitry (e.g., microcontroller 172), which manages the operations of the controller 100. As described above, the controller 100 includes communication circuitry (circuitry for communicating with the IPG 10 and/or other devices via FSK and/or other short-range RF protocols) that is used to send and receive data to/from the IPG 10 and/or other devices. Wireless data transfer between the controller 100 and the IPG 10 takes place in generally the same way as described above with respect to communications between the IPG 10 and a traditional controller 40. When data is to be sent from the controller 100 to the IPG 10 via FSK link 42, coil 185 is energized with alternating current (AC), which generates a magnetic field, which in turn induces a voltage in the IPG's telemetry coil 32. The generated magnetic field is FSK modulated (180) in accordance with the data to be transferred. The induced voltage in coil 32 can then be FSK demodulated (82) at the IPG 10 back into the telemetered data signals. Data telemetry in the opposite direction via FSK link 42 from the IPG 10 to the controller 100 occurs similarly.

The controller 100 additionally includes communication circuitry for communicating via short-range RF protocols (e.g., Bluetooth, WiFi, Zigbee, MICS, etc.). Specifically, the controller 100 includes short-range RF modulation and demodulation circuitry 190 and 192 for communicating via antenna 195. Such short-range RF communications may enable connection of the controller 100 to another consumer electronics device 300 carried by the patient, such as a smartphone. In one embodiment, the controller 100 may include control circuitry that enables the controller 100 to act as a communications bridge between the consumer electronics device 300 and the IPG 10 such that a patient can additionally control the IPG 10 via an application executed on the device 300. For example, the controller 100 may operate to convert communications received from the device 300 via the short-range RF protocol into communications understandable by the IPG 10 via the link 42. Even when the IPG includes short-range RF communication circuitry (such as IPG 10′ in FIG. 2B) that would enable it to communicate directly with the device 300, the controller 100 (typically manufactured by the manufacturer of the IPG 10) may provide a level of security over communications between the IPG 10 and the third-party device 300. For example, the controller 100 may receive a communication from the consumer electronics device 300 that is intended to be forwarded to the IPG 10, and the controller 100 may determine whether the communication is compliant with one or more rules that prevent the IPG 10 from being put into an unsafe state (e.g., a command that would significantly increase stimulation amplitude, etc.). If the communication from the consumer is electronics device 300 is compliant with the one or more rules, it may be forwarded to the IPG 10, but, if the communication is not compliant with the one or more rules, the controller 100 may prevent the communication from being forwarded to the IPG 10. While data telemetry between the IPG 10 and the controller 100 is depicted and described as occurring via near-field inductive coupling using FSK modulation, it will be understood that such communications may also occur via short-range RF protocols as described above, in which case FSK modulation and demodulation circuitry 180 and 182 and coil 185 may be omitted.

The microcontroller 172 monitors the status of the mechanical buttons 130, 132, and 134 and the mode switch 136 as well as input from the display 110 if it is configured as a touchscreen display. The microcontroller 172 also receives input from a motion sensor 175 that is positioned within the controller's housing 120. Although the controller 100's control circuitry is described as a microcontroller 172, the control circuitry may include any programmable control device such as a microprocessor or digital signal processor. The control circuitry may also be implemented as a custom designed circuit that may be embodied in hardware devices such as application specific integrated circuits (ASICs) and field programmable gate arrays (FPGAs). The motion sensor 175 may include one or more accelerometers and/or gyroscopes, and the input from the motion sensor 175 enables the microcontroller 172 to evaluate the movement and orientation of the controller 100. The motion sensor 175 may additionally include a global positioning satellite (GPS sensor). The microcontroller 172 additionally receives input from the sensor 140. As described above, the sensor 140 may generate one or more signals that are indicative of various physiological parameters of the patient. The status of any of the various inputs (whether generated by components local to the controller 100 or received from the IPG 10) may be stored in a memory 179 that is accessible by the microcontroller 172. The memory 179 may include one or more non-transitory computer-readable storage mediums such as Electrically Programmable Read-Only Memory (EPROM) or Electrically Erasable Programmable Read-Only Memory (EEPROM) used by the microcontroller 172. The memory 179 may be used to tangibly retain computer program instructions or code associated with the various applications that are executable by the microcontroller 172 to perform the functions of the controller 100. Such program code includes the program code that enables control of the IPG 10, program code that enables the controller 100 to operate as a watch (e.g., display time, date, and other information in accordance with user-selected watch interfaces), and program code associated with the above-described smartwatch applications (e.g., applications for receiving and displaying text messages and emails, receiving and displaying news and weather reports, and accessing and displaying traffic and navigation information). The program code stored in the memory 179 may, for example, cause the microcontroller 172 to determine whether the controller 100 is operating in the first mode of operation as a standalone IPG controller or in the second mode of operation and to provide a first interface (e.g., an IPG controller interface) when it is determined that the controller 100 is being used in the first mode of operation and a second interface (e.g., a smartwatch interface) when it is determined that the controller 100 is being used in the second mode of operation.

Based on the status of the various inputs, the microcontroller 172 (or a separate graphics processor) generates the video output to the display 110. Because operation of the display 110 consumes a significant amount of power, the input from the motion sensor 175 may be utilized to determine when video output should be sent to the display 110. For example, the video signal may only be output to the display following a detected movement of the controller 100 to an orientation in which it is likely to be viewed by the patient (e.g., an orientation in which the display 110 is positioned upwards). The microcontroller 172 can also send an output to the motion generator 177, which causes the controller 100 to pulse or vibrate, based on the status of the various inputs. The motion generator 177 may be an unbalanced electric motor or a linear actuator, for example. The microcontroller 172 may issue a control signal to the motion generator 177 to alert the patient of a particular condition. For example, the motion generator 177 may be utilized to alert the patient of a fault or low battery condition of the IPG 10. In one embodiment, the motion generator 177 may only be utilized when the mode switch 136 (or the mode switch in combination with selected signals from the sensor 140) indicates that the controller is being utilized in the second mode of operation (i.e., when the controller 100 is positioned against the patient's skin and the patient is therefore likely to perceive the motion of the controller 100).

Because the controller 100 may typically be affixed to the patient's wrist (with the sensor 140 positioned against the patient's skin), the inputs received by the microcontroller 172 from the sensor 140 and the motion sensor 175 provide information about the patient that can be utilized to provide closed-loop control of the IPG 10 without initiation by the patient. That is, the microcontroller 172 may cause the controller 100's communication circuitry to communicate one or more stimulation parameters to the IPG 10 based on signals from the sensor 140 and/or the motion sensor 175. For example, as described above, the sensor 140 can generate signals that are indicative of the patient's blood oxygen saturation and/or heart rate, skin temperature, oxygen uptake, respiration, motor neuron activity, and/or voice properties. From these signals, the microcontroller can derive information regarding the patient's sleep patterns, respiratory status, body activity, and level of pain, which information can be utilized to adjust one or more stimulation parameters of the IPG 10 without manual input from the patient. For example, when the signals from the sensor 140 indicate a change in the level of the patient's pain, the controller 100 may adjust the amplitude of stimulation that is being provided. In addition, when the controller 100 is connected to an Internet server, some or all of this closed-loop control processing may be offloaded to the server. That is, the patient's physiological parameters may be communicated to a remote Internet sever where they are evaluated. The server may then communicate back information (e.g., a level of pain based on the parameters) that can be utilized by the controller 100 to adjust one or more stimulation parameters of the IPG 10.

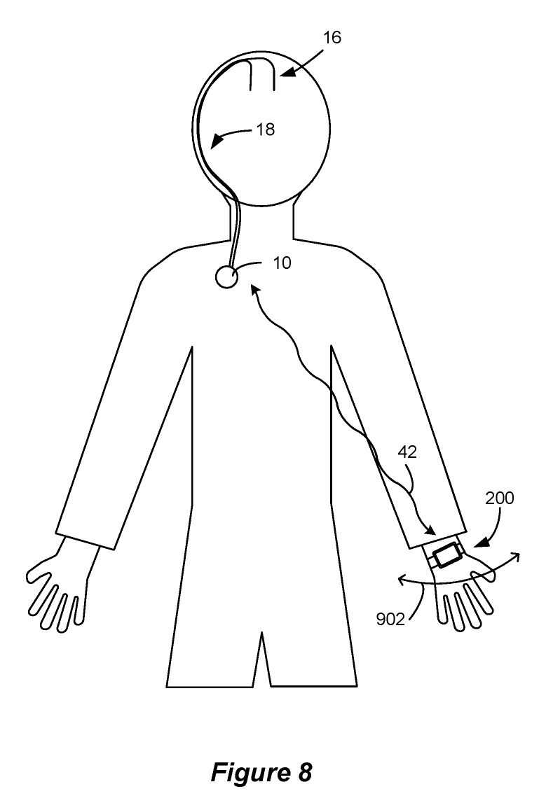

Similarly, when the controller 100 is affixed to the patient's wrist or other body part, the motion sensor 175 provides information about the patient's movement that can be used to adjust the parameters of the stimulation provided by the IPG 10. For example, as illustrated in FIG. 8, when the IPG 10 is utilized in a Deep Brain Stimulation (DBS) application, the motion sensor 175 in the controller 100 may be utilized to detect a tremor condition. More specifically, the microcontroller 172 may execute a movement algorithm that assesses the signals generated by the motion sensor 175 to determine whether the signals represent a periodic movement, and, if so, the amplitude and frequency of the periodic movement. If the movement algorithm detects that the movement is indicative of a tremor, the microcontroller 172 may generate a feedback signal to be sent to the IPG 10. The feedback signal may specify the measured parameters that are indicative of tremor (e.g., the frequency and amplitude of the periodic movement) so that the IPG 10 can make its own control adjustment. Alternatively, the feedback signal may be an instruction to make a specific adjustment to the therapy, such as increasing or decreasing the stimulation amplitude, or it may be an entire stimulation program, specifying all stimulation parameters to be used by the IPG 10. The use of a motion sensor for providing closed-loop control of DBS is described in U.S. Pat. No. 9,119,964, which is incorporated herein by reference in its entirety.

The motion sensor 175 can also be utilized to adjust stimulation therapy based on patient movements in applications other than DBS. For example, the motion sensor 175 (perhaps in combination with the sensor 140) may be utilized to detect a patient's activity, such as running, walking, sitting, or sleeping. For SCS applications, such changes in activity are a primary reason that a patient may desire to adjust the type of stimulation to achieve a more complete management of pain. Thus, based on an activity determined as a result of signals generated by the sensor 140 and the motion sensor 175, the controller 100 may adjust the stimulation settings of the IPG 10 without initiation by the user. For example, the controller may select a different pre-configured stimulation program or may adjust the stimulation amplitude of the current stimulation program based on the determined activity. In one embodiment, the controller 100 may alert the patient of the intent to change the stimulation settings (e.g., via the display 110 and/or the motion generator 177) to receive a user confirmation of the changed stimulation settings before the settings are communicated to the IPG 10. In one embodiment, the above types of closed-loop control may only be enabled when the controller 100 determines that it is being used in the second mode of operation (e.g., when the mode switch 136 indicates that a band is connected).

While the miniaturized controller 100 has been described as being connectable to a watchband such that it may function as a smartwatch 200, the controller 100 may additionally or alternatively be utilized in different types of wearable configurations. FIG. 9 illustrates the controller 100 mated with a band 230 to be worn as a necklace 240. FIG. 10 illustrates the controller 100 mated with a band 250 to be worn as an anklet 260. When the controller 100 is converted to an accessory other than a smartwatch 100, the controller 100's functionality may differ from the functionality provided in the smartwatch configuration. For example, rather than displaying a watch interface 154 as it does when connected to the watchband 220, the controller 100 may display a picture or other ornamental design when it is utilized in a different wearable configuration such as a necklace 240 or anklet 260. While these other configurations may not offer the same functionality as the smartwatch configuration, they still enable the patient to carry the controller 100 in a way that is convenient and in a way that enables the controller 100 to measure the patient's movements (via motion sensor 175) and physiological parameters (via sensor 140) for possible closed-loop control of the IPG 10.

It will be understood that while a particular configuration for mounting the controller 100 to the watchband 220 has been described and illustrated, other mounting configurations are possible. For example, the watchband 220 may be formed as two separate components that are each slid into a receptacle along an edge of the controller 100. In such an embodiment, the mode switch 136 may be positioned within one of the edge receptacles, or separate mode switches may be positioned in each of the edge receptacles. Furthermore, while a particular configuration of the controller 100 has been illustrated, other configurations are also possible. For example, the controller may be configured with more or fewer buttons that are positioned in different locations than the illustrated locations. Thus, while the invention herein disclosed has been described by means of specific embodiments and applications thereof, numerous modifications and variations could be made thereto by those skilled in the art without departing from the scope of the invention set forth in the claims.

What is claimed is:

1. A controller, comprising;
a housing that is configured to be attachable to a watchband; and
communication circuitry that is configured to enable communication between the controller and an implantable medical device (IMD), wherein the controller is configured to operate as a controller of the IMD in a first mode of operation and to be coupled to the watchband and worn on a patient's wrist in a second mode of operation.

2. The controller of claim 1, further comprising a display.

3. The controller of claim 2, further comprising control circuitry that is configured to provide a watch interface on the display.

4. The controller of claim 1, further comprising a sensor that is configured to be positioned against the patient's skin when the controller is worn on the patient's wrist.

5. The controller of claim 4, wherein the sensor is configured to measure at least one of the patient's heart rate, the patient's blood oxygen saturation, the patient's skin temperature, the patient's oxygen uptake, the patient's respiration, or the patient's motor neuron activity.

6. The controller of claim 1, wherein the communication circuitry enables wireless communications between the controller and a base station.

7. The controller of claim 1, wherein the communication circuitry enables wireless communications between the controller and a consumer electronics device.

8. The controller of claim 7, further comprising control circuitry that enables the controller to function as a communications bridge between the IMD and the consumer electronics device.

9. The controller of claim 8, wherein the control circuitry is configured to:
receive a communication from the consumer electronics device that is intended to be forwarded to the IMD;
determine whether the communication from the consumer electronics device is compliant with one or more rules;
forward the communication to the IMD when the control circuitry determines that the communication is compliant with the one or more rules; and
prevent the communication from being forwarded to the IMD when the control circuitry determines that the communication is not compliant with the one or more rules.

10. The controller of claim 1, further comprising:
one or more sensors; and
control circuitry that is configured to cause the communication circuitry to communicate one or more stimulation parameters to the IMD based, at least in part, on one or more signals from the one or more sensors.

11. The controller of claim 10, wherein the one or more sensors comprise one or more motion sensors, and wherein the control circuitry is configured to cause the communication circuitry to communicate the one or more stimulation parameters when the one or more signals from the one or more motion sensors are indicative of patient tremor.

12. The controller of claim 10, wherein the control circuitry is configured to determine whether the controller is being used in the second mode of operation.

13. The controller of claim 12, wherein the control circuitry is configured to cause the communication circuitry to communicate the one or more stimulation parameters to the IMD only when the controller is being used in the second mode of operation.

14. The controller of claim 1, wherein the housing has a volume of less than or equal to 1.1 cubic inches.

15. A system, comprising:
a controller, comprising:
a housing having one or more first connectors; and
communication circuitry that is configured to enable communication between the controller and an implantable medical device (IMD); and
a watchband having one or more second connectors that are configured to be attachable to the one or more first connectors, wherein the controller is configured to operate as a controller of the IMD in a first mode of operation and to be coupled to the watchband and worn on a patient's wrist in a second mode of operation.

16. The system of claim 15, wherein the one or more first connectors comprise at least one switch that indicates whether the watchband is attached to the controller.

17. The system of claim 15, wherein the controller further comprises a sensor that is configured to be positioned against the patient's skin when the controller is worn on the patient's wrist.

18. The system of claim 17, wherein the sensor is configured to measure at least one of the patient's heart rate, the patient's blood oxygen saturation, the patient's skin temperature, the patient's oxygen uptake, the patient's respiration, or the patient's motor neuron activity.

19. The system of claim 15, wherein the controller further comprises:
one or more sensors; and
control circuitry that is configured to cause the communication circuitry to communicate one or more stimulation parameters to the IMD based, at least in part, on one or more signals from the one or more sensors.

20. The system of claim 19, wherein the one or more sensors comprise one or more motion sensors, and wherein the control circuitry is configured to cause the communication circuitry to communicate the one or more stimulation parameters when the one or more signals from the one or motion sensors are indicative of patient tremor.
